(19) United States
(12) Patent Application Publication
(10) Pub. No.: US 2007/0105119 A1
(43) Pub. Date: May 10, 2007

(54) Method for detecting analytes by means of an analyte/polymeric activator bilayer arrangement

(76) Inventors: Zhinqiang Gao, Singapore (SG); Xie Hong, Singapore (SG); Chunyan Zhang, Singapore (SG); Yuan Hong Yu, Singapore (SG)

Correspondence Address: INTELLECTUAL PROPERTY / TECHNOLOGY LAW, PO BOX 14329, RESEARCH TRIANGLE PARK, NC, 27709, US

(21) Appl. No.: 10/577,293

(22) PCT Filed: Oct. 25, 2004
(86) PCT No.: PCT/SG04/00351

§ 371 Date: Apr. 29, 2006

Related U.S. Application Data
(60) Provisional application No. 60/515,229, filed on Oct. 29, 2003.

(51) Int. Cl. C12Q 1/68; C12Q 1/26

(52) U.S. Cl. 435/6; 435/25; 205/777.5

(57) ABSTRACT

The invention relates to the field of analytical sensors. In particular, the invention relates to a method for the detection of analytes in a sample by means of an electrode arrangement, which is characterized by the formation of a conductive bilayer of analytes and an agent for increasing the conductivity of said analytes on the surface of an electrode. The invention is also directed to an electrode arrangement useful for performing such method as well as to the use of such electrode arrangement as biosensor. Also disclosed is a novel class of redox polymers that are suitable for being used in the electrochemical detection of analytes. A method of making this class of polymers is also disclosed.

The invention relates to the field of analytical sensors. In particular the invention relates to a method for the detection of analytes in a sample by means of an electrode arrangement, which is characterized by the formation of a conductive bilayer of analytes and an agent for increasing the conductivity of said analytes on the surface of an electrode. The invention is also directed to an electrode arrangement useful for performing such method as well as to the use of such electrode arrangement as biosensor.

### BACKGROUND THE INVENTION

The detection and quantification of analytes such as macromolecular biopolymers is a fundamental method not only in analytical chemistry but also in biochemistry, food technology or medicine. To date, the most frequently used methods for determining the presence and concentration of biopolymers include the detection by autoradiography, fluorescence, chemiluminescence or bioluminescence as well as electrochemical techniques (reviewed in, e.g., in Bakker, E. and Telting-Diaz. M. (2002) Anal. Chem. 74, 2781-2800).

However, autoradiography cannot be applied in many fields due to the use of hazardous radiochemicals, whereas optical detection methods usually involve tedious labeling procedures as well as expensive reagents and technical equipment. Electrochemical detection techniques, on the other hand, have become an attractive alternative in view of both high sensitivity and low costs.

With regard to the detection of nucleic acid molecules three major electrochemical detection approaches are currently applied, namely conductivity measurements (Park, S. J. et al. (2002) Science 295, 1503-1506), nucleic acid-intercalation methods (Zeman, S. M. et al. (1998) Proc. Natl. Acad. Sci. USA 95, 11561-11565; Erkkila, K. E. et al. (1999) Chem. Rev. 99, 2777-2795), and detection by catalytic amplification (Caruana, D. J. & Heller, A. J. (1999) J. Am. Chem. Soc. 121, 769-774; Patolsky, F. et al. (2002) Angew. Chem. Int. Ed. 41, 3398-3402).

Park et al., supra, have reported a DNA array detection method using oligonucleotides functionalized with gold nanoparticles. The detection limit of 500 fM has been found, which is, however, not sufficient to identify very rare nucleic acid species encoding, for example, transcription factors or certain cell-surface receptors. Nucleic acid-intercalation methods are often hampered by a low signal-to-noise ratio, since most DNA-intercalating agents do not only intercalate into double-stranded DNAs (dsDNA) but also bind to single-stranded DNA molecules via electrostatic interactions, even though to a much lower extent. However, an improved ferrocene labeled naphthalene diimide threading intercalator that binds to dsDNA more selectively (but not exclusively) has been synthesized (Takenaka, S. et al. (2000) Anal. Chem. 72, 1334-1341).

Current advances in DNA bioelectronics have focused on the employment of nucleic acid/enzyme-conjugates as bioelectrocatalysts (Caruana & Heller, supra; Patolsky et al., supra). Similarly, nucleic acid functionalized liposomes or nanoparticles were used as particulate labels for the amplification of the DNA sensing processes. Very recently, a detection limit of 0.5 μl has been reported for a 38-base oligonucleotide using an enzyme-amplified detection method that corresponds to approximately 3000 molecules (Zhang, Y. et al. (2003) Anal. Chem., page EST 2.4). However, this sensitivity is only achieved when analyzing short DNA oligonucleotides, usually 20-50 bases in length. The use of these methods for the detection of larger nucleic acid molecules such as genomic DNAs has turned out to be difficult due to high background signals, which results in a rather low sensitivity in pM or even nM range.

Thus, there remains a need for an alternative method for the detection of analytes, which overcomes the above limitations and allows the detection even of macromolecular analytes with high sensitivity.

### SUMMARY OF THE INVENTION

In one aspect, the invention provides a method for the electrochemical detection of an analyte molecule by means of an detection electrode, the method comprising:

(a) immobilizing capture molecules, which are capable of binding the analyte molecule to be detected, on the detection electrode;

(b) contacting the detection electrode with a solution supposed to contain the analyte molecule to be detected;

(c) allowing the analyte molecule contained in said solution to bind to the capture molecules on the detection electrode, thereby allowing formation of complexes between a capture molecule and an analyte molecule, said complexes forming a first layer on the electrode;

(d) contacting the detection electrode with an electrochemical activator, wherein said electrochemical activator has an electrostatic net charge that is complementary to the electrostatic net charge of the complex formed between a capture molecule and an analyte molecule, thereby forming a second layer on the electrode, wherein the second layer and the first layer together form a conducting bilayer;

(e) contacting the detection electrode with an agent capable of transferring electrons to or from the electrochemical activator from or to the electrode, respectively;

(f) performing an electrical measurement at the detection electrode;

(g) detecting the analytes by comparing the result of the electrical measurement obtained with that of a control measurement.

In another aspect, the inventions provides an electrode arrangement, comprising a detection electrode useful for carrying out an electrochemical detection of an analyte molecule as disclosed herein, comprising:

(a) a first layer on the detection electrode comprising complexes between a capture molecule, which is capable of binding the analyte molecule to be detected, and an analyte molecule; and

(b) a second layer comprising an electrochemical activator, wherein said electrochemical activator has an electrostatic net charge that is complementary to the electrostatic net charge of the complex formed between a capture molecule and an analyte molecule, wherein the second layer and the first layer together form a conducting bilayer.

In yet another aspect, the invention provides a biosensor for the electrochemical detection of an analyte molecule, comprising:

(a) a detection electrode

(b) a first layer on the detection electrode comprising complexes between a capture molecule, which is capable of binding the analyte molecule to be detected, and an analyte molecule; and

(c) a second layer comprising an electrochemical activator, wherein said electrochemical activator has an electrostatic net charge that is complementary to the electrostatic net charge of the complex formed between a capture molecule and an analyte molecule, wherein the second layer and the first layer together form a conducting bilayer.

In yet another aspect, the invention provides a water soluble redox polymer comprising:

(a) a first monomer unit comprising a polymerizable ferrocene derivative; and

(b) a second monomer unit comprising an acrylic acid derivative having a (terminal) primary acid or base, acid or base functional group capable of acquiring a net charge.

In one embodiment, the acrylic acid derivative in this new water soluble redox polymer is represented by the general formula (I) 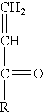
wherein R is selected from the group consisting of CₙH₂ₙn—NH₂, CₙH₂ₙ—COOH, NH—CₙH₂ₙn—PO₃H, and NH—CₙH₂ₙn—SO₃H, wherein the alkyl chain can be optionally substituted, and wherein n is an integer from 0 to 12.

In yet another aspect, the invention provides a process for preparing a water soluble, redox polymer, said process comprising:

polymerizing a first monomer unit comprising a polymerizable ferrocene derivative with a second monomer unit comprising an acrylic acid derivative having an acid or base functional group capable of acquiring a net charge, wherein said polymerization is carried out in an aqueous alcoholic medium.

### BRIEF DESCRIPTION OF THE DRAWINGS

The invention will be better understood with reference to the detailed description when considered in conjunction with the non-limiting examples and the drawings, in which:

FIG. 1 depicts a schematic illustration of the detection method according to the present invention. First, as shown in FIG. 1a the capture molecules 20, which are capable of binding the analyte to be detected, are immobilized on the surface of the detection electrode 10. Optionally, a blocking agent 15 may be added—either individually or together with the capture molecules—in order to occupy free binding sites on the electrode surface and thus to reduce the background signals. Then, the detection electrode is exposed to a solution supposed to contain the target analyte 30. The analytes molecules are allowed to bind to the capture molecules forming a first layer on the surface of the detection electrode. Subsequently, the electrochemical activator 40 as well as an agent 50 for transferring electrons to or from the electrochemical activator from or to the electrode, respectively, are brought in contact with the electrode surface (in arbitrary order or as mixture). The electrochemical activator has an electrostatic net charge that is complementary to the electrostatic net charge of the complex formed between a capture molecule and an analyte molecule, thereby forming a second layer on the electrode, wherein the second layer and the first layer together form a conducting bilayer. In the optional presence of substrate molecules 55, the current generated from the catalytic oxidation of the substrate are detected amperometrically. The current directly correlates to the target analyte concentration in the sample solution. FIG. 1b illustrate the use of linker molecules in order to modify the surface of the detection electrode (e.g. a gold electrode).

FIG. 2 depicts a representative gelelectrophoretic separation of PCR products encoding full-length rat TP53 cDNA (lanes 1-3) and full-length GAPDH cDNA (lanes 4-6) using different ratios of biotin-dUTP/dTTP. Lane M, DNA size maker. Lanes 1-3 correspond to biotin-16-dUTP/dTTP ratios of 0:100, 35:65, and 65:35, respectively. Lanes 4-6, correspond to biotin-21-dUTP/dTTP ratios of 0:10, 1:10, and 2:10, respectively.

FIG. 3 depicts cyclic voltamograms of a gold electrode (a) coated with a mixed self-assembled monolayer in a 2.5 mM K₃Fe(CN)₆ and 0.50 M Na₂SO₄, (b) with DNA/redox polymer bilayer in the PBS, and (c) with DNA/redox polymer bilayer in the 2.5 mM K₃Fe(CN)₆ and 0.50 M Na₂SO₄. Scan rate: 100 mV/s. The current scale in (b) was multiplied by a factor of 10 for the sake of clarity.

FIG. 4 depicts cyclic voltamograms of a gold electrode after hybridization with the GAPDH cDNA in PBS (curve a) and 20 mM glucose solution (curve b) with (A) a capture probe complementary to the GAPDH cDNA, and (B) a capture probe non-complementary to the GAPDH cDNA. Scan rate: 10 mV/s.

FIG. 5 depicts the amperometric responses of a gold electrode after hybridization with the GAPDH cDNA in the PCR mixture (a) with a capture probe complementary to the GAPDH cDNA, and (b) with a capture probe non-complementary to the GAPDH cDNA. Working potential: 0.36 V, 40 mM glucose.

FIG. 6 depicts the amperometric responses of a gold electrode after hybridization with 50, 100, 200, and 500 fM TP53 cDNA in 2.5 μl droplets, respectively. Working potential: 0.36 V, 40 mM glucose.

FIG. 7 depicts the amperometric responses of a gold electrode after hybridization with a mixture of E. coli 16S rRNA, E. coli 23S rRNA, and full-length rat GAPDH cDNA. Curve (a) corresponds to the response of E. coli 16S rRNA, curve (b) to the response of rat GAPDH cDNA, whereas curve (c) represents a blank control. 1 μl droplets were used. Working potential: 0.35 V, 60 mM glucose.

FIG. 8 depicts the amperometric responses of a gold electrode after hybridization of an E. coli 16S rRNA-specific DNA capture probe with 200 fM of (a) a fully complementary synthetic oligonucleotide, (b) a one-base mismatched oligonucleotide, and (c) a two-base mismatched oligonucleotide, respectively in 1 μl droplets in order to evaluate the sensitivity of the assay system. Working potential: 0.35 V, 60 mM glucose.

FIG. 9 depicts the dependence of the oxidation current from the analyte concentration. GAPDH cDNA capture probes are immobilized on the surface of a gold electrode and contacted with 10 μM biotinylated GAPDH cDNA. Following hybridization, a glucose oxidase/avidin-conjugate is attached via avidin-biotin interaction. Finally, a redox polymer is brought to the electrode surface through layer-by-layer electrostatic self-assembly. Glucose detection medium: PBS (pH 7.4). Working potential: 0.35 V.

FIG. 10 shows a schematic diagram of the coupling redox reaction, which takes places in a redox polymer mediated biosensor.

FIG. 11 illustrates a structure of the basic unit of a water-soluble and cross-linkable polymer of the present invention. The figure shows a repeating unit found in a copolymer of vinylferrocene and an acrylic acid derivative.

FIG. 12 depicts the general reaction equation in the co-polymerization reaction of vinyl ferrocene and an acrylic acid derivative.

FIG. 13 shows a Fourier Transform Infra Red (FT-IR) spectrum of the redox polymers PAA-VFc and PAAS-VFc produced according to a process of the invention.

FIG. 14 shows a ultra-violet (UV)-visible spectrum of Fc, PAA, PAAS and the co-polymers obtained from co-polymerization with VFc.

FIG. 15 shows cyclic voltamograms of redox polymers In various systems. Phosphate-buffered saline was used, and the potential scan rate applied in obtaining the voltamograms was 100 mV/s.

FIG. 16 shows another cyclic voltamogram of a redox polymer PAA-VFc that is cross-linked with glucose oxidase-bovine serum albumin (GOx-BSA) film on gold electrode. Phosphate-buffered saline was used, and the potential scan rate applied in obtaining the voltamograms was 50 mV/s.

### DETAILED DESCRIPTION

The invention is based on the finding that the sensitivity of the detection of analytes, such as biopolymers (which are generally nonconductive or only poorly conductive), can be significantly improved by the use of an electrochemical activator that is present in solubilized form and whose net charge in solution is complementary (i.e. opposite) to the net charge of the analyte molecules to be detected or a complex comprising the same. Due to their opposite charges, the analytes and complexes comprising the same form together with the electrochemical activator a very stable bilayer via electrostatic layer-by-layer self-assembly. This bilayer functions as an “electron-exchange bridge” (or “electron shuttle”) across the complete surface of the electrode, which influences the current flow at the electrode used for the detection of the analyte. The use of the bilayer has also the advantage of providing a larger and more homogenous contact area to the electrode, which also contributes to the increased sensitivity of the detection method of the invention compared to other procedures known in the art.

The term “detecting” according to the present invention refers to both qualitative and quantitative detection of analytes in a sample, meaning that the term “detecting” also includes determining the absence of an analyte in the sample. By use of the present method, analyte concentrations as low as about 1 fM (i.e. 10⁻¹⁵ M) can be unambiguously detected. The range of concentrations of analyte suitable for detection in the inventive method is about 10⁻¹² M to 10⁻¹⁵ M. The upper concentration limit of analyte for carrying out the detection is usually about 10⁻¹¹ M. It is noted in this respect that is the clear that in case the analyte is believed to be present in a sample in an amount higher than 10⁻¹¹ M, than the sample can be diluted such that it the amount of thereof is within the sensitivity range of the present invention.

The term “capture molecule” as used herein may refer to a single type of molecules, for example a single-stranded nucleic acid probe with a defined nucleic acid sequence. However, the capture molecules may also comprise different types of molecules, for example nucleic acid probes having different nucleic acid sequences (which therefore also exhibit different binding specificities). The capture molecules may also be antibodies or other types of proteinaceous binding molecules such as the class of anticalins® (polypeptides which exhibit, like antibodies, specific binding characteristics for a given ligand (cf. also Beste et al. (1999) Proc. Natl. Acad. Sci. USA 96, 1898-1903), which recognize different surface regions (epitopes) of a proteinaceous compound. The use of different types of capture molecules does not only allow the simultaneous or consecutive detection of different analytes such as two or more genomic DNAs, each of them having binding specificity for one particular type of capture molecule, but also the detection of the same analyte via different recognition sequences, e.g., the 5′- and 3′-termini of a nucleic acid molecule or two ligand binding sites of a receptor molecule, which enhances the likelihood to detect even a few copies of an analyte in a sample.

The term “electrochemical activator” as used herein refers to any compound that is capable of activating the agent that transfers electrons between the analyte and the electrode, that binds (preferably specifically) to the analytes to be detected, and that exhibits a conductivity for electric current, which is higher than that of said analyte.

In one embodiment of the invention, the electrochemical activator is a polymeric redox mediator. In some embodiments of the invention, the electrochemical activator contains redox-active metal ions. Examples of such metal ions are silver, gold, copper, nickel, iron, cobalt, osmium or ruthenium ions or mixtures thereof, all of which can bind as cations to negatively charged groups on the surface of the analytes to be detected by electrostatic interaction. For example, if the analytes to be detected are nucleic acids, such cations bind to the negatively charged phosphate backbone of said nucleic acids. If proteins are to be detected, such cations may bind to the side chains of acidic amino acids, such as aspartate or glutamate.

In general, suitable polymeric redox mediators should have a chemical structure, which prevents or substantially reduces the diffusional loss of the redox species during the period of time that the sample is being analyzed. One type of such a non-releasable polymeric redox mediator comprises a redox species covalently attached to a polymeric compound. Such redox polymers typically are transition metal compounds, wherein a redox-active transition metal-based pendant group is covalently bound to a suitable polymer backbone, which on its own may or may not be electroactive itself. Examples of this type are poly(vinyl ferrocene) and poly(vinyl ferrocene co-acrylamide). Alternatively, the polymeric redox mediator may comprise an ionically-bound redox species. Typically, these mediators include a charged polymer coupled to an oppositely charged redox species. Examples of this type include a negatively charged polymer such as Nafion® (Dupont) coupled to a positively charged redox species such as an osmium or ruthenium polypyridyl cation or vice versa a positively charged polymer such as poly(1-vinyl imidazole) coupled to a negatively charged redox species such as ferricyanide or ferrocyanide. Furthermore, the redox species can also be coordinatively bound to the polymer. For example, the redox mediator may be formed by coordination of an osmium or cobalt 2,2′-bipyridyl complex to poly(1-vinyl imidazole) or poly(4-vinyl pyridine). Another example is poly(4-vinyl pyridine co-acrylamide) coordinated with an osmium 4,4′-dimethyl-2,2′-bipyridyl complex. Useful redox mediators as well as methods for their synthesis are described in U.S. Pat. Nos. 5,264,104; 5,356,786; 5,262,035; 5320,725; 6,336,790; 6,551494; and 6,576,101.

In a further embodiment of the invention, the electrochemical activator is selected from the novel class of redox polymers that is described in detail later herein. Briefly, this novel class of redox polymers comprises poly(vinyl ferrocene), poly(vinyl ferrocene)-co-acrylamide, poly(vinyl ferrocene)-co-acrylic acid, and poly(vinyl ferrocene)-co-acrylamido-(CH₂)ₙ-sulfonic acid, and poly(vinyl ferrocene)-co-acrylamido-(CH₂)ₙ-phosphonic acid, wherein n is an integer from 0 to 12.

The term “agent capable of transferring electrons” as used herein refers to any agent, which upon activation by the electrochemical activator is able to transfer electrons to or from the electrochemical activator from or to the electrode, respectively. That is an agent capable of donating and re-accepting electrons, resulting in a decrease or an increase of the oxidation state of at least one atom of said agent. Thereby, the agent is bound to, intercalated in or associated with the conducting brayer formed of the analyte/capture molecule-complexes and the electrochemical activator molecules, respectively.

The agent of transferring electrons may only serve this purpose. It is however, also possible that the agent capable of transferring electrons simultaneously functions as capture molecule. This is in particular the case, when the analyte to be detected is an enzyme substrate the conversion of which can be detected by an electric measurement (cf., Example 2).

In one embodiment of the invention, the agent capable of transferring electrons is an enzyme or an enzyme-conjugate. Usually, any enzyme may be used that leads to the generation of an detectable electric current. The enzyme may be selected from the group of oxidoreductases. Examples of suitable oxidoreductases are glucose oxidase, hydrogen peroxidase, lactate oxidase, alcohol dehydrogenase, hydroxybutyrate dehydrogenase, lactic dehydrogenase, glycerol dehydrogenase, sorbitol dehydrogenase, glucose dehydrogenase, malate dehydrogenase, galactose dehydrogenase, malate oxidase, galactose -oxidase, xanthine dehydrogenase, alcohol oxidase, choline oxidase, xanthine oxidase. choline dehydrohenase, pyruvate dehydrogenase, pyruvate oxidase, oxalate oxidase, bilirubin oxidase, glutamate dehydrogenase, glutamate oxidase, amine oxidase, NADPH oxidase, urate oxidase, cytochrome C oxidase, and actechol oxidase.

The analytes to be detected by the inventive method may be nucleic acids, oligonucleotides, proteins, peptides or complexes thereof, such as DNA/protein- or RNA/protein-complexes. The analyte may also be an oligo- or a polysaccharide or a low molecular weight chemical compound that exhibits in free or conjugated form which exhibits features of an immunological hapten. Examples of such compounds are small molecule drugs, nutrients, pesticides or toxins, to name a few.

In one preferred embodiment of the invention, the analytes to be detected are nucleic acid molecules. Thereby, the term “nucleic acids or nucleic acid molecules” as used herein refers to genomic DNA, cDNA as well as RNA molecules. The term “oligonucleotides” according to the present invention refers to smaller nucleic acid molecules (DNA and RNA) of approx. 10 to 80 base pairs (bp) in length, with molecules of 15 to 40 bp in length being preferred. The nucleic acids may be double-stranded but may also have at least one single-stranded region or may be present completely in the form of single strands, for example due to previous thermal denaturation or another kind of strand separation for their detection. In a preferred embodiment of the invention, the sequence of the nucleic acids to be detected is pre-defined, i.e. is known, wherein the complete sequence may be known or at least a part thereof. Because of the high sensitivity of the detection method of the invention, the nucleic acid molecules to be detected can be derived from a genomic sample and may be present in a low copy number, medium copy number or high copy number.

Suitable capture molecules for the detection of nucleic acids according to the inventive method are nucleic acid probes, i.e. single-stranded DNA or RNA molecules. Probes having a sequence that is partially or fully complementary to the single-stranded region of the respective target nucleic acid are preferably used. The nucleic acid probes may be synthetic oligonucleotides or longer nucleic acid sequences, as long as the latter do not fold in any structure preventing hybridization of the probe with the nucleic acid to be detected. Also preferred as capture molecules are nucleic acid probes that comprise modified nucleotides such as nucleotides carrying a biotin-, digoxigenin- or thiol-label. It is, however, also possible to use DNA- or RNA-binding proteins or agents as capture molecules.

In another preferred embodiment of the invention, the analytes to be detected are proteins or peptides. These may be composed of the 21 naturally occurring amino acids (including selenocystein) but may also contain, for example, amino acid residues modified by sugar residues or any type of posttranslational modification. By means of the inventive method, it may also be possible to detect complexes of nucleic acids and proteins, such as complexes of a RNA-binding protein with its cognate RNA target or of a transcription factor with its respective DNA-binding domain.

Preferred capture molecules for the detection of proteins or peptides are any type of ligands with binding activity for the proteins or peptides to be detected. Examples of such ligands are low molecular-weight enzyme agonists or antagonists, receptor agonists or antagonists, pharmaceuticals, sugars, antibodies or any molecule capable of specifically binding proteins or peptides.

The capture molecules, irrespective of the analyte for which they have binding activity, can be immobilized on the detection electrode by any suitable physical or chemical interaction. These interactions include, for example, hydrophobic interactions, van der Waals interactions, or ionic (electrostatic) interactions as well as covalent bonds. This further means that a capture molecule can be directly be immobilized on the surface of the electrode by hydrophobic interaction, van der Waals interactions or electrostatic interaction or through covalent coupling using a linker molecule should the surface of the electrode not be suitable for direct immobilization. It is also possible to use as linker molecule a molecule that has binding activity for the capture molecule and then immobilize the capture molecule by binding to that linker molecule by non covalent interactions, i.e. complex formation (cf. Example 2, wherein glucose oxidase molecules serve as capture molecules).

The method according to the invention may be carried out by using virtually any electrode arrangement known in the art that comprises a detecting or working electrode. Such a electrode arrangement usually also comprise a counter electrode as well as a reference electrode. The detecting electrode may be a conventional metal electrode (gold electrode, silver electrode etc.) or an electrode made from polymeric material or carbon, the surface of which has been optionally modified in order to facilitate the immobilization of the capture molecule. The electrode arrangement that comprise the detecting electrode may also be a common silicon or gallium arsenide substrate, to which a gold layer and a silicon nitride layer have been applied, and which has subsequently been structured by means of conventional lithographic and etching techniques to generate the electrode arrangement(s). In structuring, the distance between the detecting and the counter electrodes may vary, depending on the kind of structuring technique used and the type of analytes to be detected. The distance between the electrodes is generally from about 50 μm to 1000 or several 1000 μm.

The method according to the present invention also allows detecting more than one type of analyte simultaneously or consecutively in a single measurement. For this purpose, a substrate comprising a plurality of electrode arrangements as disclosed herein may be used, wherein different types of capture molecules, each of which exhibiting (specific) binding affinity for a particular analyte to be detected, are immobilized on the electrodes of the individual electrode arrangements. Alternatively, it may also be possible to use a plurality of electrode arrangements, each of which being provided with only one type of capture molecules.

An example of an electrode arrangement which may be used for carrying out the inventive method, is a conventional interdigitated electrode. Consequently, an arrangement provided with a plurality of interdigitated electrodes, i.e. an electrode array, can be employed for parallel or multiple determinations. Another usable electrode arrangement is an electrode arrangement in the form of a trench or a cavity, which is formed, for example, by holding regions such as, for example, a gold layer on which the capture molecules capable of binding the analytes are immobilized being located on two opposite side walls.

The present method comprises, as a first step, the immobilization of the capture molecules capable of binding the analyte to be detected on the surface of the electrode. The capture molecules may be immobilized by any conventional technique known in the art. If multiple analyses are performed, the capture molecules may be applied, for example, with the aid of inkjet printing techniques.

Optionally, a blocking agent may be added—either individually or together with the capture molecules—in order to reduce the background signals. When added individually, the blocking agent may be added in advance of the ample solution or after the electrode (coated with the capture molecules) has been contacted with the sample solution in order to prevent those capture molecules, which are not bound to analyte molecules, from interacting with the electrochemical activator in an unspecific manner. Any agent that can be immobilized on the electrode and that is able to prevent (or at least to significantly reduce) the interaction between the capture molecules and the analyte molecules is suitable for that purpose. Examples of such agents are thiol molecules, disulfides, thiophene derivatives, and polythiophene derivatives. One particular useful class of blocking reagents used in the invention are thiol molecules such as 16-mercaptohexadecanoic acid, 12-mercaptododecanoic, 11-mercaptodecanoic acid or 10-mercaptodecanoic acid.

A solution, for example an electrolyte, supposed to contain the analyte molecule to be detected is then contacted with the electrode such that the analyte molecule can bind to the capture molecules forming a first layer on the electrode surface. If the solution contains a plurality of different analytes to be detected, the conditions are chosen so that said analytes can either bind simultaneously or consecutively to their respective capture molecules.

After allowing the analyte molecule to bind to the capture molecules, unbound capture molecules may be removed from the electrode. Removing the unbound capture molecules is optional but may often be advantageous, since certain capture molecules (e.g., oligonucleotides) are capable of binding not only the analytes to be detected but also the agents for increasing the conductivity of said analytes (e.g., reducible metal cations), which will certainly interfere with the results of the electrochemical measurements.

The unbound capture molecules may be removed enzymatically. In the case, the capture molecules are DNA probes, this may be accomplished by an enzyme, which selectively breaks down single-stranded DNA, such as mung bean nuclease, nuclease P1 or nuclease S1. If the capture molecules are low molecular-weight ligands, these ligands are immobilized on the electrodes via an enzymatically cleavable covalent linkage, for example via an ester linkage. In this case, it is possible to use, for example, a carboxyl ester hydrolase (esterase) in order to remove unbound ligand molecules. This enzyme selectively hydrolyzes ester linkages between the electrode and unbound ligand molecule. In contrast, the ester linkages between the electrode and ligand molecules bound by peptides or proteins remain intact due to reduced sterical accessibility of the linkage.

The detection electrode, on which the analytes to be detected are immobilized via specific capture molecules is then contacted with the electrochemical activator, which is allowed to bind to said analytes and imparts to these electrical conductivity. The electrochemical activator has a electrostatic net charge that is complementary to the electrostatic net charge of the complex formed between a capture molecule and an analyte molecule, thereby forming a second layer on the electrode, wherein the second layer and the first layer together form a stable conducting bilayer via electrostatic self-assembly.

Furthermore, the detection electrode is contacted with an agent capable of transferring electrons to or from the electrochemical activator from or to the electrode, respectively, which may facilitate or even amplify the electron transfer between the analyte and the electrode. The agent capable of transferring electrons may be added simultaneously with the electrochemical activator, in advance of contacting the electrode arrangement with the electrochemical activator or after the same has already been bound to the electrode arrangement. Any agent capable of transferring electrons may be used that upon activation by the electrochemical activator (and optionally in the presence of substrate molecules) is able to transfer electrons to or from the electrochemical activator. Thereby, the agent is bound to, intercalated in or associated with the conductive brayer formed on the electrode surface. In a preferred embodiment of the invention, the agent is an enzyme or an enzyme-conjugate. The layer-by-layer configuration of the conductive brayer structure significantly reduces or even eliminates the non-specific adsorption and electrostatic interaction of the agents capable of transferring electrons, thus resulting in a higher signal-to-noise ratio and higher detection limits.

Subsequently, an electrical measurement is performed at the detection electrode. Electrical measurements according to the invention include measurements of current as well as of voltage. The result obtained is then compared to that of a control measurement, in which capture molecules unable to bind the analyte to be detected are used. Examples of such “control” capture molecules are nucleic acid probes having a sequence not complementary to that of the target nucleic acid molecule or a low molecular-weight ligand unable to interact with the receptor molecule to be detected. If the two electrical measurements, i.e. “sample” and “control” measurement, differ in such a way that the difference between the values determined is greater than a pre-defined threshold value, the sample solution contained the relevant analytes to be detected.

The method may also be designed in such a way that a reference measurement and a measurement for detecting analytes are performed simultaneously. This may be done, for example, by carrying out a reference measurement only with the control medium and, at the same time, a measurement with the sample solution supposed to contain the analytes to be detected.

The present invention is also directed to an electrode arrangement, comprising a detection electrode suitable for carrying out an electrochemical detection of an analyte molecule as disclosed herein, comprising:

(a) a first layer immobilized on the detection electrode comprising complexes between a capture molecule, which is capable of binding the analyte molecule to be detected, and an analyte molecule; and

(b) a second layer comprising an electrochemical activator, wherein said electrochemical activator has an electrostatic net charge that is complementary to the electrostatic net charge of the complex formed between a capture molecule and an analyte molecule, wherein the second layer and the first layer together form a conducting bilayer.

In one preferred electrode arrangement of the invention, the electrochemical activator forming part of the conducting bilayer on the detection electrode is a polymeric redox mediator capable of transferring electrons between the analyte and the electrode. More preferred are electrode arrangements, wherein the electrochemical activator contains metal ions, and in particular preferred embodiments these metal ions are selected from the group consisting of silver, gold, copper, nickel, iron, cobalt, osmium, ruthenium, and mixtures thereof.

In one embodiment of the invention, the electrode arrangement further comprises an agent capable of transferring electrons to or from the polymeric redox mediator from or to the electrode, respectively, wherein the agent is bound to, intercalated in or associated with the conducting bilayer on the detection electrode. In a preferred electrode arrangement according to the invention, the agent is an enzyme or an enzyme-conjugate.

The detection electrode as well as the corresponding electrode arrangement of the invention may be used as biosensor. Such sensors are needed in many fields such as analytical chemistry, biochemistry, pharmacology, microbiology, food technology or medicine in order to analyze the presence and concentration of certain analytes in a given sample. For example, biosensors may be used to monitor glucose in blood or urine samples of diabetic patients or lactate during critical care events. However, such biosensors may also be employed for the detection and quantification of contaminants in drinking water, milk or any other food. Another application is the use of such biosensors in genome projects, for example, for detecting genes or gene mutations such as single nucleotide polymorphisms (SNPs) that are causative or indicative for a disease. On the other hand, such biosensors may also be used in proteomics, e.g. for the analysis of protein-protein interactions, as well as for the identification of ligands for a particular receptor molecule.

The invention is also directed to a biosensor for electrochemical detection of an analyte molecule, comprising:

(a) a detection electrode;

(b) a first layer on the detection electrode comprising complexes between a capture molecule, which is capable of binding the analyte molecule to be detected, and an analyte molecule; and

(c) a second layer comprising an electrochemical activator, wherein said electrochemical activator has an electrostatic net charge that is complementary to the electrostatic net charge of the complex formed between a capture molecule and an analyte molecule, wherein the second layer and the first layer together form a conducting brayer.

The invention also relates to new ferrocene-based redox polymers that are inter alia well suited for being used as electrochemical activator in the detection method of the invention as well as any other known electrochemical detection. Although ferrocene-containing monomers usually undergo free radical polymerization with great difficulty, the inventors have found that redox polymers containing ferrocene can be elegantly and readily prepared using an alcoholic medium prepared, for example, from a mixture of ethanol and water, together with a persulfate salt as radical initiator.

These ferrocene derivatives based polymers can be used as diffusional electron transfer mediators in homogeneous systems.

These ferrocene derivatives based polymers can also be used as mediators that are immobilized on an electrode surface and then attached to a protein molecule, such as an enzyme or an antigen, via cross-linking between cross-linkable functional groups found both in the enzyme and in a side chain of the redox polymer.

Suitable polymerizable ferrocene derivatives that can be used as a first monomer to form a redox polymer should possess a side chain unit having an unsaturated bond, such as a C—C double or triple bond, or a N—N double bond or a S—S double bond. Examples of such side chain units include alkenyl groups, represented by the general formula R₁—C═C—. The double bond can be located at any position along the carbon chain. Aromatic groups, e.g. phenyl, toluoyl, and naphthyl groups, can also be used. Furthermore, the polymerizable group can also comprise substituted C-atoms wherein a halogen (e.g. fluorine, chlorine, bromine or iodine), oxygen or hydroxyl moiety for example, substitutes one or more hydrogen atoms on carbon atoms in the group. Further examples include an alkynyl and a disulphide group.

In some embodiments of the polymer of the invention, the polymerizable ferrocene derivative is selected from the group consisting of vinyl-ferrocene, acetylene-ferrocene, styrene-ferrocene and ethylene oxide-ferrocene.

The presence of an unsaturated bond in these derivatives would allow the ferrocene molecule to be attached to a polymer backbone via co-polymerization with another species having also at least one unsaturated C—C double or triple bond, or a N—N double bond or a S—S double bond, via free radical polymerization.

For the second monomer unit that is used in co-polymerization with the polymerizable ferrocene derivative, any suitable acrylic acid derivative having an primary acid or base functional group capable of acquiring a net charge can be used. This means, the invention provides for positively as well as negatively charged polymers and thus ensure that conducting bilayers as explained above can be formed, irrespective of the net charge of the complex formed between capture molecules and analyte molecules. In general, there are two requirements for selecting a suitable acrylic acid derivative for use as a monomer. In order for it to co-polymerize with the ferrocene derivative, it should possess of at least one unsaturated bond, which can be provided by a C—C double or triple bond, or a N—N double bond or a S—S double bond for example. Secondly, the acrylic acid derivative should be able to function as a Bronsted-Lowry acid or base by producing H⁺ ions or by accepting H⁺ ions, respectively. Examples of functional groups which can provide a Bronsted-Lowry acid or base function include primary amine groups which can accept H⁺ ions to form charged amine groups, or carboxyl groups, or sulfate which can donate H⁺ ions when the acid functionalities dissociates to release H⁺ ions. In this respect, it is noted that although the use of primary amine groups is preferred in the present application, it is evident for the skilled person that also secondary or tertiary amine groups present in the acrylic acid derivative can be used in order to generate a positively charged redox polymer. In this respect, it is also noted that the acid or base functionality, although it is a primary one, does not need to be a terminal group, but in case of a branched side chain can be present “within” the shorter one of the side chains.

While any suitable acrylic acid derivative having an acid or base functional group, preferred monomers that are used as the second monomer in a redox polymer of the present sensor is an acrylic acid derivative represented by the general formula (I): 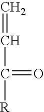
wherein R is selected from the group consisting of CₙH₂ₙ—NH₂, CₙH₂ₙ—COOH, NH—CₙH₂ₙ—PO₃H, and NH—CₙH₂ₙ—SO₃H, wherein the alkyl chain is optionally substituted, and wherein n is an integer from 0 to 12, preferably 0 to 8. Thus, the alkyl group can be straight chained or branched and can comprise double or triple bonds or a cyclic structure, such as cyclohexyl. Examples of suitable aliphatic moieties within the substituent R are methyl, ethyl, propyl, isopropyl, butyl, isobutyl, pentyl, isopentyl, hexyl, cyclohexyl or octyl, to name a few. The aliphatic group can further be substituted by an aromatic group such as phenyl, a halogen atom, a further base or acid group, or an O-alkyl group, for example. Exemplary aromatic groups that can be present as substituents are phenyl, toluoyl or naphthyl. The halogen atom can be selected from fluoride, chloride or bromide. Examples of suitable o-alkyl groups are methoxy, ethoxy, propoxy or butoxy, whereas the n-alkyl group is selected from —NHMe, —N(Me)₂, —N(Ethyl)₂ or —N(Propyl)₂.

In some embodiments, the redox polymer of the invention has a molecular weight of between about 1000 and 5000 Daltons, or preferably between about 2000 and 4000 Daltons.

The inventors have found that the amount of radical initiator affected the degree of polymerization. High amounts of radical initiator significantly reduced polymerization efficiency, resulting in redox polymers having lower molecular weight. This also meant that relatively little radical initiator was needed in the polymerization process, compared to normal free radical polymerization reactions. Apart from the quantity of free radical initiator used, the addition sequence of reactants, which is discussed in greater detail below in relation to a process of the invention, also affected polymerization efficiency.

In another embodiment of the invention, the redox polymer has a ferrocene loading of between about 2% to about 20%, typically of about 3% and about 14%.

The present invention is also directed to a process for preparing such a water-soluble redox polymer. The process essentially involves polymerizing a first monomer unit of a polymerizable ferrocene derivative with a second monomer unit comprising an acrylic acid derivative, such as a primary, secondary or tertiary acrylamide, to produce a copolymer. The acrylic acid derivative possesses an acid or base functional group capable of acquiring a net charge. Importantly, the polymerization reaction is carried out in an aqueous alcoholic medium in the presence of an initiator.

The addition sequence of the monomers and initiator can be varied. For example, it is possible to mix the first and second monomer in alcoholic medium, and then add the initiator to the initiate the reaction. It is also possible to dissolve one of the monomers in aqueous alcoholic medium first, and then add the initiator to it, before adding the other monomer to the mixture.

An alcoholic medium can be prepared with any water miscible organic alcohol, for example, aliphatic alcohols such as ethanol, or aromatic alcohols such as phenols. The volumetric ratio is usually within the range of ca 5:1 to 1:1 (alcohol/water). In some embodiments, it is about 3:1.

In an embodiment of the invention, the process is carried out using an aqueous alcoholic solvent comprising ethanol and water.

Although polymerization may proceed without the addition of an initiator, it is desirable to add an initiator, which attacks the electron-rich centers found at the unsaturated bonds in the monomers. Accordingly, in another embodiment of the invention, polymerization is initiated by adding a free radical initiator.

Any free radical initiator can be used. Examples include inorganic salts such as persulfate salts, or organic compounds such as benzoyl peroxide or 2,2′-azo-bis-isobutyrylnitrile (AIBN), which are able to produce radical fragments called initiator fragments, each of which has one unpaired electron which can function as a free radical which attack the unsaturated bonds in the monomer units.

In some embodiments of the process according to the invention, the free radical initiator is selected form the group consisting of ammonium persulfate, potassium persulphate and sodium persulfate.

In some of these embodiments of the invention, the weight ratio of free radical initiator added is between about 20 mg to 40 mg per 1 gram of monomer.

The process according to the invention can be carried out under reflux above room temperature, but generally below 100° C. In one embodiment, polymerization is carried out under reflux at a temperature between about 60° C. to 80° C.

The length of time that is required for polymerization can be dependent upon the temperature used and the amount of initiator added to the reaction broth. Typically, polymerization is carried out for a period between 10 to 40 hours, and preferably for about 24 hours.

An embodiment of the present inventive process further comprises producing a pre-reaction mixture prior to polymerizing said first and second monomers, comprising:

dissolving the acrylic acid derivative monomer unit in an aqueous alcoholic medium; then

adding the free radical initiator; and then

adding the polymerizable ferrocene derivative monomer unit to the mixture.

In a further embodiment of the above process, the feeding ratio of acrylic acid derivative to polymerizable ferrocene derivative in the pre-reaction mixture that falls between about 5% and 15% of the weight of monomer added is preferable in order to obtain a redox polymer having a suitable molecular weight and viscosity.

In yet another embodiment, the polymerizable ferrocene derivative-monomer unit is dissolved in an aqueous alcoholic medium before being added to the reaction mixture.

### EXAMPLES

### Example 1

### Detection of Nucleic Acids

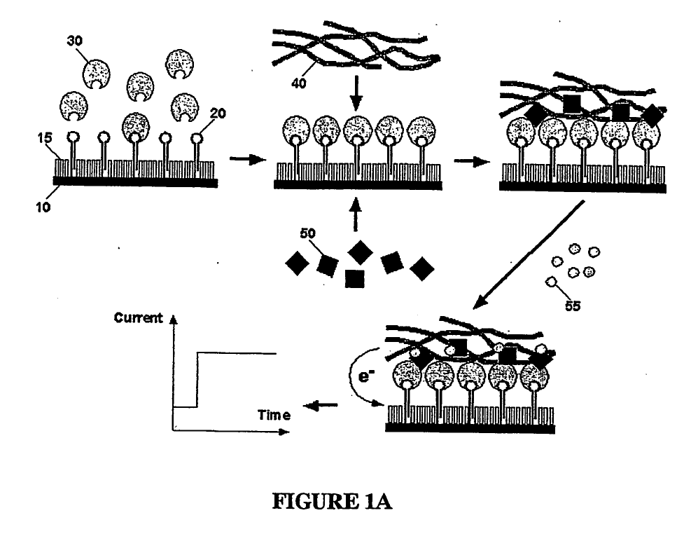

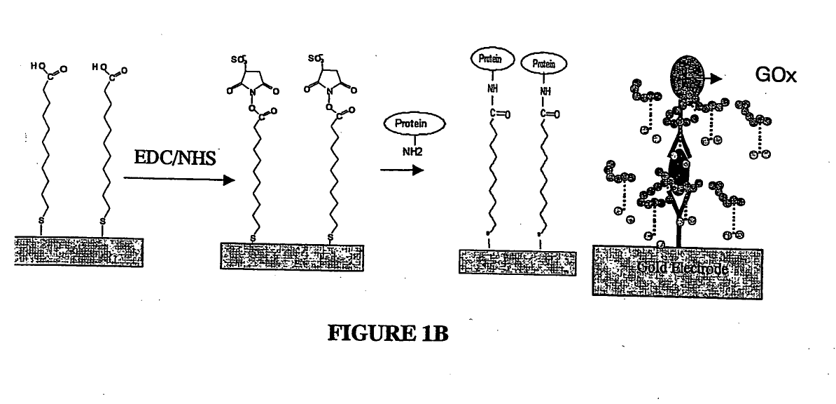

In general, the detection of nucleic acids according to the invention is performed as illustrated in FIG. 1. First, a mixture of thiolated oligonucleotides (also carrying a biotin modification as label) serving as capture molecules (20) and thiol molecules serving as blocking agent (15) for reducing the background is immobilized on a gold electrode surface (10). Then, the electrode is exposed to a solution supposed to contain the target analyte (30). Following hybridization to its complementary biotinylated target DNA (i.e. the capture molecule) an enzyme-conjugate (50) is attached via avidin-biotin interaction. Finally, a redox polymer (40) is brought to the electrode surface through layer-by-layer electrostatic self-assembly. The redox polymer layer electrochemically activates the enzyme labels bound to the target DNA. In the presence of substrate molecules (55), the current generated from the catalytic oxidation of the substrate are detected amperometrically. The current directly correlates to the target analyte concentration in the sample solution.

### Example 1.1

### mRNA Extraction from Rat Tissues and Synthesis of Biotinylated cDNA

The extraction of rat liver mRNA was performed using the Dynabeads® mRNA DIRECT™ Kit (Dynal ASA, Oslo, Norway) according to the manufacturers instructions. For reverse transcription (RT), 10 ng of this mRNA were used in a total volume of 20 μl containing 1×eAMV buffer from Sigma-Aldrich (50 mM Tris-HCl, pH 8.3, 40 mM KCl, 8.0 mM MgCl₂, 1 mM DTT), 500 μM of each dNTP, 1.0 μM anti-sense primer, 20 U RNase inhibitor, and 20 U enhanced avian myeloblastosis virus reverse transcriptase (eAMV). The samples were incubated for 50 min at 56° C. in a DNA thermal cycler (Gene Amp PCR System 9700, Applied Biosystems, Foster City, Calif., USA.) and the cDNA obtained was directly used as template for PCR amplification.

PCR was performed with 2.0 μl of the RT-reaction mixture in a total volume of 50 μl containing 1× AccuTaq buffer from Sigma-Aldrich (5 mM Tris-HCl, 15 mM ammonium sulfate, pH 9.3, 2.5 mM MgCl₂, 0.1% Tween 20), 0.40 μM of each primer, 2.5 U JumpStart AccuTaq LA DNA polymerase and 10 mM dNTP (Roche, Basel, Switzerland). Two different genes were chosen as analytes, a housekeeping gene, glyceraldehydes-3-phosphate dehydrogenase (GAPDH), and a regulated tumor protein gene 53 (TP53).

The following primers were used: GAPDH sense, 5′-ATGGTGMG GTCGGTGTCAA-3′ (SEQ ID NO: 1); GAPDH anti-sense, 5′-TTACTCCTTGGA GGCCATGT-3′ (SEQ ID NO: 2); TP53 sense, 5′-ATGGAGGATTCACAGTC GGA-3′ (SEQ ID NO: 3), and TP53 anti-sense, 5′-TCAGTCTG AGTCAGGCCC-3′ (SEQ ID NO: 4).

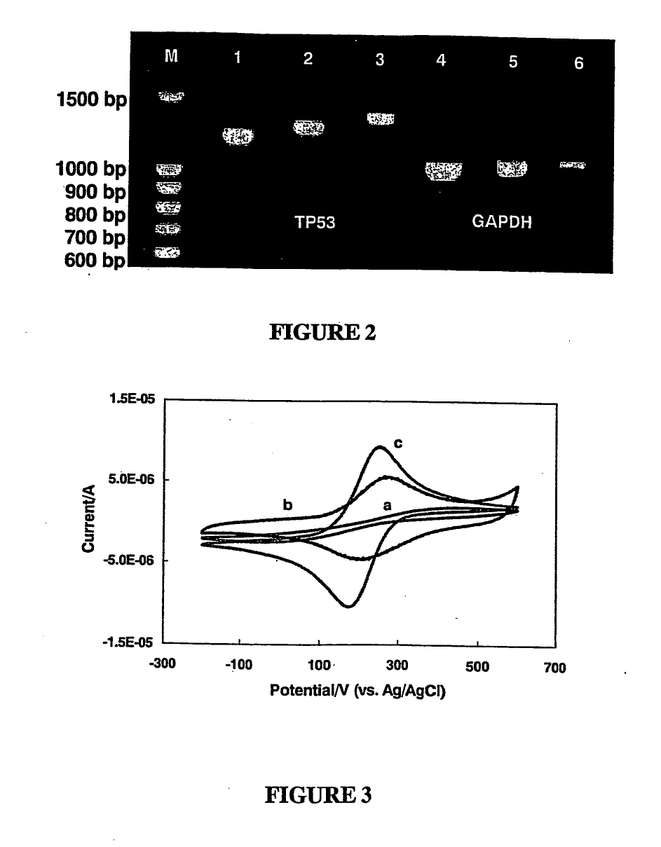

For the synthesis of biotinylated cDNA s, different amounts of biotin-16-dUTP (Roche, Germany) or biotin-21-dUTP (Clontech, Palo Alto, USA) were added to the reaction. Amplification was performed using the following profile: after an initial denaturation step at 95° C. for 5 min, 35 cycles of amplification at 95° C. for 30 s, 55.5° C. for 1 min, and 72° C. for 2 min were performed. A final extension step of 10 min at 72° C. was included to ensure synthesis of full-length DNA strands. After amplification, the PCR products were separated on a 1.0% agarose gel and visualized by staining with ethidium bromide (FIG. 2).

In FIG. 2, lanes 1 and 4 illustrate control experiments without the addition of biotin-dUTP. The PCR-fragments amplified are in good agreement with the size of the full-length rat TP53 (lane 1, 1176 bp) and GAPDH (lane 4, 1002 bp) genes, respectively. For labeling, different amounts of biotin-modified nucleotide were mixed with dNTPs and added to the PCR reaction mixture in order to examine labeling efficiency (cf. lanes 2 and 3 for TP53 as well as lanes 5 and 6 for GAPDH, respectively). The higher the ratio of biotin-16-dUTP (or biotin-21-dUTP)/dTTP, the stronger the fragment is retarded in the gel. However, with increasing ratios of biotin-modified nucleotide to normal nucleotide amplification efficiency is reduced, presumably due to the bulky side chains of biotin-modified nucleotides.

### Example 1.2

### Capture Probe Immobilization and Evaluation of Monolayer Quality

Prior to the detection of a DNA analyte, a mixture of thiolated oligonucleotides, served as capture probes, and thiol molecules were immobilized onto the gold electrode surface through self-assembly. To minimize non-hybridization related uptake of the target DNA, anionic thiol molecules were used to form the blocking component of the mixed monolayer. The following capture probes were used: for the detection of GAPH, 5′-T₁₂TTACTCCTTGGA GGCCATGTAGG-3′ (SEQ ID NO: 5); and 5′-T₁₂ATG GTGMGGTCGGTGTCA ACGG-3′ (SEQ ID NO: 6); for the detection of TP53, 5′-T₁₂ATGGAGGATTCAC AGTCGGA-3′ (SEQ ID NO: 7) and 5′-T₁₂TCAGTCTGAGTCAGGCCCCA-3′ (SEQ ID NO: 8); and as a control, 5′-T₁₂CCTCTCGCGAGTCAACAGAMCG-3′ (SEQ ID NO: 9). The oligonucleotides were thiolated at their 5′-termini using 11-mercaptoundecanoic acid according to standard procedures and assembled on the gold electrodes via exposing clean electrodes in 50 μM oligonucleotide solutions for 3-16 hours. The remaining surface was then blocked with 11-mercaptoundecanoic acid (MUA).

The formation of the mixed self-assembled monolayer on gold electrode was routinely monitored by optical ellipsometric, contact angle and surface coverage measurements. All the data obtained indicate a single compact mixed molecular layer coated on the gold electrode. As expected, the obvious pathway of electron transfer between the monolayer coated electrode and the electro-active species in solution would be via electron tunneling across the insulating monolayer. The electron tunneling barrier characteristics of the capture probe monolayer and the mixed monolayer were investigated by cyclic voltametry in a 0.50 M Na₂SO₄ solution containing 2.5 mM ferricyanide (FIG. 3). As shown in FIG. 3a, irreversible voltametric waves for Fe(CN)₆^{3-/4-} with a very large peak-to-peak potential separation, >400 mV at 100 mVs⁻¹, compared with a value of 59 mV for a reversible process obtained at a bare gold electrode were observed at the mixed monolayer coated gold electrode indicating that the monolayer impedes electron transfer between the electrode and the solution. The redox currents, which are mainly caused by electron tunneling across the monolayer, were significantly reduced and lost its reversible character. A poly(vinylpyridine-co-acrylamide) copolymer partially pyridine-complexed with an Os(4,4′-dimethyl-2,2′-bipyridine)₂Cl^{+/2+} (PVP-PAA-Os) was used as redox polymer (Gao, Z. et al. (2003) Angew. Chem. Int. Ed. 41, 810-813). However, since the redox polymer is positively charged and the electrode is negatively charged, a brief soaking of the electrode in the 5.0 mg/ml PVP-PAA-Os solution, resulted in the formation of a DNA/redox polymer bilayer on the electrode via layer-by-layer electrostatic self-assembly. As illustrated in FIG. 3b, the brayer coated electrodes exhibited exactly as expected for a highly reversible surface immobilized redox couple with little change after exhaustive washing with water and PBS and after numerous repetitive potential cycling between −0.4 V and +0.8 V, revealing a highly stable surface immobilized electrostatic bilayer on gold electrode. Such results ascertain that all of the osmium redox centers are allowed to reach the electrode surface and proceed to reversible heterogeneous electron transfer. The total amount of bound osmium redox centers, 1.8-8.0×10⁻¹⁰ mole/cm², which depends on the amount of anionic species (nucleic acid and enzyme label) and the amount of nucleic acid bound to the electrode, was estimated from the area either of the oxidation peak or the reduction current peak. Subsequent voltametric tests in the ferricyanide solution showed results identical to that obtained at the bard gold electrode (FIG. 3c). These changes are attributed to the decrease in electron tunneling due to bilayer formation. The presence of anionic species in the film did not appreciably alter the electrochemistry of the redox polymer.

### Example 1.3

### GAPDH cDNA Hybridization and Detection

In a preliminary hybridization test, the PCR amplification mixture was used as analyte without further purification. Biotinylated GAPDH cDNA (cf. Example 1.1) was used as target and TE (10 mM Tris-HCl, 1.0 mM EDTA) containing 0.10 M NaCl as hybridization buffer. Prior to hybridization, the target cDNA was denatured at 95° C. for 5 min and cooled on ice. Hybridization was carried out in a 55° C. water bath for 30 min, wherein GAPDH cDNA was selectively bound by the complementary capture probe and thus immobilized on the surface of the electrode. Repeated washing steps with the hybridization buffer removed all unspecific nucleic acids. Then, the electrode was exposed to 2.5 μl glucose oxidase/avidin D-conjugate (GOx-A, 5 mg/ml; Vector Laboratories, San Diego, Calif., USA) at 35° C. for 30 min. After 3 washing steps with the PBS buffer to remove excess enzyme labels the electrode was exposed for at least 10 min to a 2.5 μl PVP-PAA-Os redox polymer solution and rinsed with PBS buffer.

Electrochemical measurements were carried out in a Faraday cage with a low-noise CH Instruments Model 660A electrochemical workstation (CH Instruments, Austin, Tex., USA) in conjunction with a Pentium computer. Cyclic voltametry was conducted in both the PBS buffer and the PBS buffer containing 20 mM glucose. An Ag/AgCl electrode (Cypress Systems, Lawrence, Kans., USA) was used as the reference electrode and a platinum wire as the counter electrode. Amperometric measurements were carried out at 0.36 V. All potentials reported in this report were referred to the Ag/AgCl reference electrode.

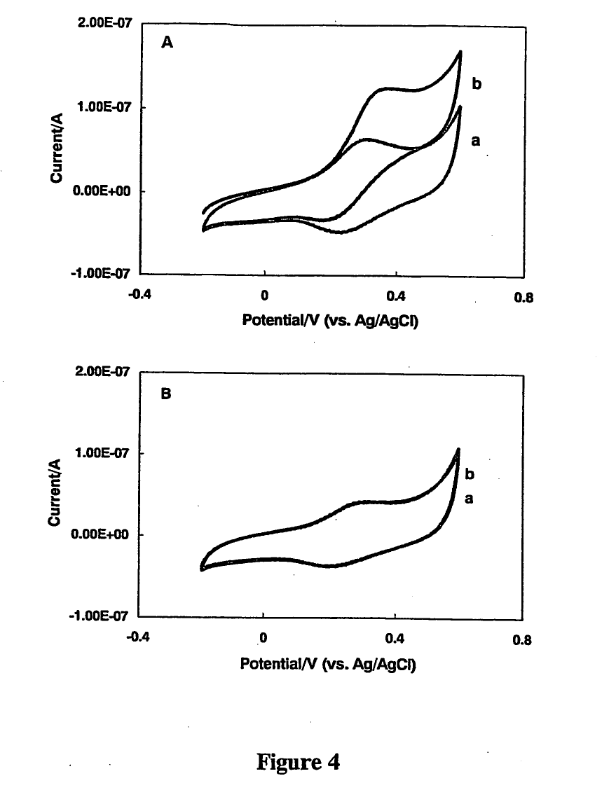

Typical cyclic voltamograms of the electrode hybridized with the target analyte are shown in FIG. 4. FIG. 4A is a voltamogram of the electrode with capture probe complementary to GAPDH cDNA in the PBS buffer (curve a) and in a 20 mM glucose solution (curve b) after hybridization. Obvious catalytic current was observed in the presence of glucose due to the presence of glucose oxidase in the bilayer. In contrast, non-complementary probes failed to capture any GAPDH cDNA from the PCR mixture and thus no enzyme labels were able to bind to the electrode surface resulting in no detectable catalytic current (FIG. 4B, curves a and b, respectively).

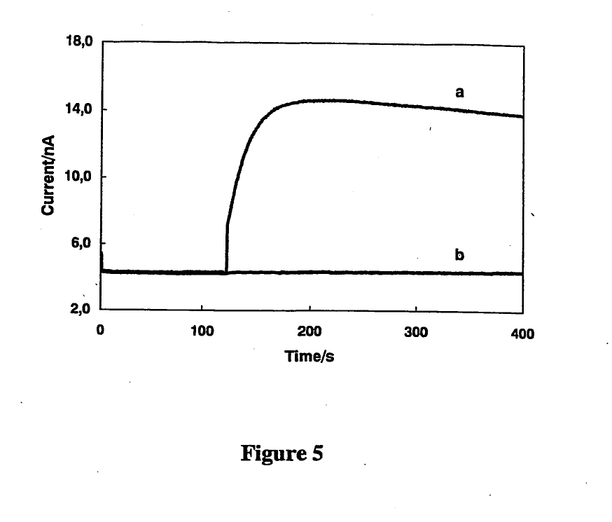

When immersing the electrode assembly in the PBS buffer, the oxidation current in amperometry increased 10.2 nA at 0.36 V (vs. Ag/AgCl) upon adding 40 mM glucose to the buffer (FIG. 5). In control experiment using non-complementary capture probes a negligible change of current was observed. The amperometric results complimented the cyclic voltametric data and confirmed again that the GAPDH cDNA was successfully detected from the PCR mixture with high specificity. Under optimized experimental conditions, a dynamic range was found between 2.0 fM and 1.0 pM with a detection limit of 0.50 fM.

### Example 1.4

### Detection of Rat TP53 cDNA

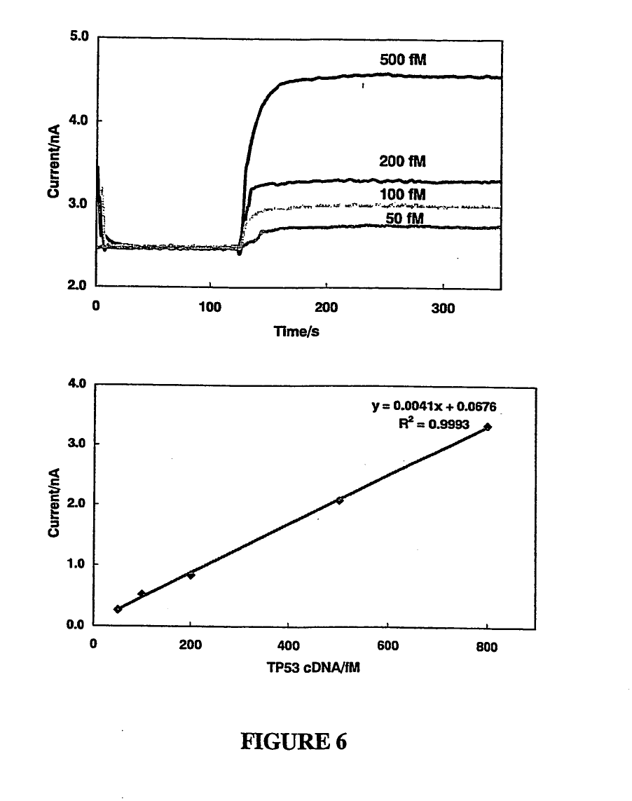

Biotinylated rat TP53 cDNA was synthesized as described in Example 1. After PCR amplification, the total amount of TP53 cDNA was determined to be 17.2 ng/μl (22.5 pM). Samples containing 10, 50, 100, 200, 500 and 800 fM TP53 cDNA (diluted in TE buffer) were analyzed. The TP53-specific cDNA in the PCR mixture was immobilized on the surface of the electrode by its complementary capture probes before adding enzyme labels and redox polymer, respectively (cf. Example 1.3). A catalytic current was detected at 0.36 V, which directly corresponds to the amount of the TP53 cDNA. As depicted in FIG. 6, the current increased linearly with the concentration of the TP53 cDNA within this range. The detection limit was found at about 1.0 fM. Taking into consideration the sample volume, as few as 1500 copies of TP53 DNA molecules were successfully detected using the proposed approach. To our knowledge, this is the lowest amount of genomic DNA detected electrochemically reported so far.

### Example 1.5

### Detection of Nucleic Acids in a Nucleic Acid Mixture

The nucleic acid biosensor was applied to the detection of E. coli 16S rRNA as well as GAPDH cDNA in a mixture containing: 0.5-1500 fM E. coli 16S rRNA, 100-5000 fM E. coli 23S rRNA, 0.2-2000 fM full-length rat GAPDH cDNA, 1-500 mM BSA, and 1-100 mM salmon sperm DNA. The GAPDH cDNA was prepared by isolation rat liver mRNA and subsequent PCR amplification as described in Example 1.1. The total amount of GAPDH cDNA obtained was 5.0±0.5 μg. Afterwards, the PCR product was diluted by factor 10⁶ with a pH 8.0 Tris-EDTA buffer.

The following probes were used: E. coli 16S rRNA-specific capture probe: 5′-GCCAGCGTTCAATCTGAGCCATGATCAAACTCTTCAAAAA AAAAAAAAA-3′ (SEQ ID NO: 10); E. coli 16S rRNA-specific detection probe: 5′-AAAAAAAAAAAAAAGCTGCCTCCCGTAGGAGT-3′ (SEQ ID NO: 11). The immobilization of the capture probe on the gold electrode was performed as described in Example 1.2.

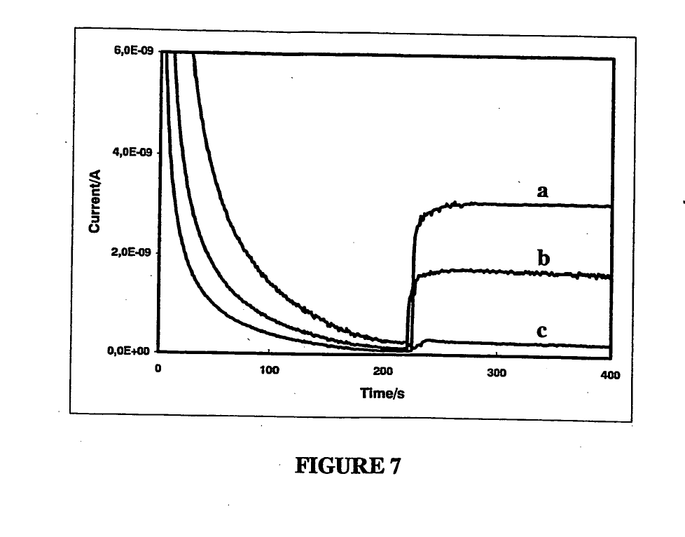

Direct hybridization and electrochemical detection were carried out on the E. coli RNA samples (cf. Examples 1.3 and 1.4, respectively). After glucose oxidase and the redox polymer were introduced, catalytic current was detected at 0.35 V, which directly corresponds to the amount of nucleic acid. As a control, non-complementary capture probes were immobilized on the electrode surface. The amperometric responses were found to be 2.95 nA for E. coli 16S rRNA and 1.65 nA for GAPDH cDNA, respectively, corresponding to a concentration of 290 fM E. coli S16 rRNA and 150 fM GAPDH cDNA (FIG. 7). These results are in good agreement with the values obtained by gel electrophoretic analysis (310 fM E. coli S 16 rRNA and 160 fM GAPDH cDNA; data not shown).

### Example 1.6

### Selectivity of the Detection System

The selectivity of the biosensor was evaluated using the above capture probe: 5′-GCCAGCGTTCAATCTGAGCCATGATCAAACTCTTC AAAAAAAAAAAAAAAA-3′ (SEQ ID NO: 10) as well as the following synthetic oligonucleotides: complementary 5′-AAATTGAAGAGTTTGATCATGGCTCAGA TTGAACGCTGGCAAAAAAAAACTCCTACGGGAGGCAGC-3′ (SEQ ID NO: 12); single-base mismatched 5′-AAATTGAAGAGTTTGATCATGTCTCAGA TTGAACGCTGGCAAAAAAAAAAAAAACTCCTACGGGAGGCAGC-3′ (SEQ ID NO: 13); two-base mismatched 5′-AAATTGAAGAGTATGATCATGTCTCAGAT TGAACGCTGGCAAAAAAAAAAAAAACTCCTACGGGAGGCAGC3′ (SEQ ID NO: 14) (nucleotide variations are shown in bold and underlined). The capture probe was immobilized on the gold electrode as described in Example 1.2.

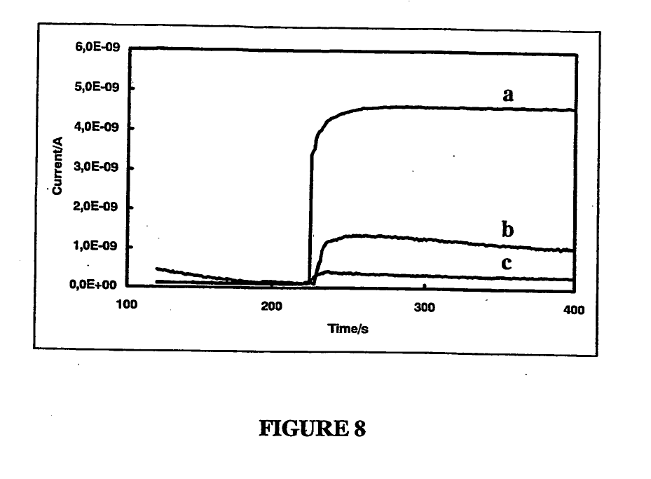

Hybridization was performed in 1 μl droplets using the 200 fM solutions of the three different DNA oligonucleotides under hybridization conditions in favor of the perfectly matched sequence (cf. Examples 1.3 and 1.4, respectively, with the exception that a hybridization temperature of 53° C. was used). The amperometric responses obtained are summarized in FIG. 8. The current increment upon adding 60 mM glucose to the detection medium of the perfectly matched sequence was 4.3±0.4 nA (curve a), whereas 1.0±0.3 nA and 0.3±0.1 nA were detected for the one-base mismatched (curve b) and two-base mismatched sequences (curve c), respectively. Thus, the biosensor readily allows discrimination between the perfectly matched and mismatched DNA oligonucleotides.

### Example 2

### Detection of a Small (Low Molecular Weight) Enzyme Substrate

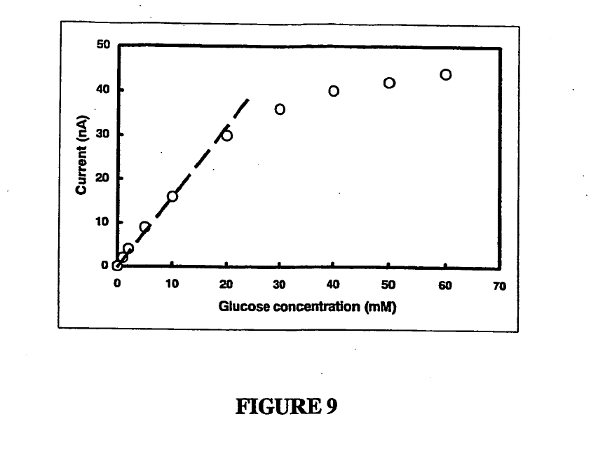

In order to evaluate the dependence of the oxidation current from the analyte concentration, a saturating amount of GAPDH cDNA capture probes were immobilized on the surface of a gold electrode and contacted with 10 μM biotinylated complementary GAPDH cDNA. Following hybridization, a glucose oxidase/avidin-conjugate is attached via avidin-biotin interaction. Finally, a redox polymer is brought to the electrode surface through layer-by-layer electrostatic self-assembly. PBS (pH 7.4) is used as detection medium at a working potential of 0.35 V. As illustrated in FIG. 9, up to about 20 mM glucose, there is a linear relationship between the oxidation current obtained and the amount of analyte detected.

In should be noted in this respect, that the brayer setting used in this Example is exactly the same as in Example 1. However, when detection of a nucleic acid as in Example 1 is intended, very high glucose concentration may be used in the method of the present invention, in order to ‘saturate’ the enzyme, or in other words, very high glucose oxidation rate may be used, to have sufficient sensitivity. When instead of the detection of the nucleic acids is intended, the detection of an enzyme substrate of an oxidoreductase is desired, this can be achieved by working with a very high nucleic acid concentration, saturating capture probes with complementary nucleic acid and an oxidoreductase such as glucose oxidase. Since the current is due to oxidation of glucose in solution (or the oxidation of the enzyme substrate in general), a current-concentration relationship exists that can be used for the detection of glucose or the oxidizable enzyme substrate in general. It is further noted that in Example 2 the glucose oxidase molecules serve as capture molecules and at the same time as agent that is capable of transferring electrons to or from the electrochemical activator from or to the electrode, respectively. Thus, Example 2 illustrates the detection method of the invention, wherein the capture molecules are (also) capable of transferring electrons to or from the electrochemical activator from or to the electrode, respectively.

### Example 3

### Detection of a Polypeptide

The detection of proteins (in analogy to nucleic acids, cf. Example 1) is performed as outlined in FIG. 1a and FIG. 1b. In this case, the electrode may be first coated with thiol molecules (e.g. 16-mercaptohexadecanoic acid), which in this case serve a linker molecules for the covalent attachment of the capture molecules. The coated electrode is then immersed in a mixture of 1-ethyl-3-(3-dimethyl aminopropyl)carbodiimide/N-hydroxy-succinimide (EDC/NHS) in order to activate the carboxylic acid groups of the linker, which will then form a covalently bond with the amino groups of the capture molecule. For example, the capture molecule can be an antibody or a low molecular weight ligand having binding affinity for a proteinaceous analyte. Then the electrode is contacted with the solution suspected to contain the analyte allowing the formation of complexes between the capture molecules and the analyte molecules. After that, a redox polymer, to which an enzyme label is attached, is brought to the electrode surface through layer-by-layer electrostatic self-assembly (cf. FIG. 1a). In the presence of substrate molecules, the current generated from the catalytic oxidation of the substrate are detected amperometrically. The current directly correlates to the target analyte concentration in the sample solution. It this also possible as shown in FIG. 1b to carry out the detection in a manner that resembles a sandwich-ELISA. For this purpose, the complexes comprising an antibody as the capture molecule and the analyte are contacted with a second antibody that has binding affinity to the analyte as well. This second antibody may be conjugated with an enzyme such as glucose oxidase that acts as the agent capable of transferring electrons to or from the electrochemical activator from or to the electrode, respectively. Thereafter, a redox polymer, to which an enzyme label is attached, is brought into contact with the electrode surface, thereby forming a layer-by-layer electrostatic self-assembly (cf. FIG. 1a) and allowing the detection of the polypeptide.

### Example 4

### Detection of a Low Molecular Weight Ligand

Using the “sandwich-ELISA like” procedure explained in Example 3, in which an antibody is used as capture molecule and the complex of this capture antibody with the analyte is contacted with a second antibody conjugated to a suitable enzyme, it is clear that virtually any small ligand such as drugs (e.g. cocaine, morphium), nutrients (saccharose, amino acids, etc.), environmentally harmful products (pesticides such as triazines, DDT, etc.) can be detected by means of the present invention.

### Example 5.1

### Synthesis of Poly(Vinylferrocene-Co-Acrylamide), Poly (Vinylferrocene-Co-Acrylic Acid), and Poly(Vinylferrocene-Co-Acrylamido-Sulphonic Acid) Copolymers

Glucose oxidase (GOx, EC 1.1.3.4, from Aspergillus niger, 191 units/mg) was purchased from Fluka (CH-9470 Buchs, Switzerland). Ferrocene (Fc), Vinylferrocene (VFc), acrylamide (AA), acrylic acid (AC), 2-Acrylamido-2-methyl-1-propanesulfonic acid (“acrylamido-sulfonic acid”, AAS, catalogue number 28,273) and persulfate salts were purchased from Sigma-Aldrich (St. Luis, Mo., USA.). All other chemicals such as acetone, ethanol, and phosphate buffered saline used were of certified analytical grade. All solutions that were used were prepared with deionized water.

UV spectra of polymers produced in the experiment was performed and recorded on an Agilent 8453 UV-visible spectrophotometer. Molecular weights were determined with a Toyo Soda high performance gel permeation chromatography in water and standard poly(ethylene oxide) and poly(ethylene glycol) for calibration.

i) Synthesis of Poly(Vinylferrocene-Co-Acrylamide) Polymers

Three samples containing 1.0 g acrylamide dissolved in 10 ml of mixture solvent of ethanol/water (3 parts to 1 part) were prepared. A 0.30 ml aliquot of 0.10 g/ml oxygen-free persulfate solution was added to each sample after being deoxygenated for 10 minutes. 3 amounts of vinylferrocene ranging from 0.05 g to 0.16 g were dissolved in degassed ethanol to form 3 vinylferrocene solution samples, the amount of ferrocene that is added for each sample being calculated to obtain acrylamide-to-vinylferrocene feeding ratios (w/w) of 95:5, 90:10 and 85:15, respectively. Each vinylferrocene sample was then added to an acrylamide-initiator mixture. Reaction mixtures were refluxed at 70° C. for 24 hours in nitrogen atmosphere. After cooling, the reaction mixtures were, separately, added drop-wisely to rapidly stirred acetone in order to precipitate a redox polymer. The precipitated redox polymer was washed with acetone and purified by multiple water-dissolving acetone-precipitating cycles. The purified product was then dried under vacuum at 50° C.

ii) Synthesis of Poly(Vinyl Ferrocene-Co-Acrylic Acid) Polymers

Three samples containing 1.0 g acrylic acid dissolved in 10 ml of mixture solvent of ethanol/water (3 parts to 1 part) were prepared. A 0.30 ml aliquot of 0.10 g/ml oxygen-free persulfate solution was added to each sample after being deoxygenated for 10 minutes. Three amounts of vinylferrocene ranging from 0.05 g to 0.16 g were dissolved In degassed ethanol to form 3 vinylferrocene solution samples, the amount of vinylferrocene that is added for each sample being calculated to obtain acrylamide-to-vinylferrocene feeding ratios (w/w) of 95:5, 90:10 and 85:15, respectively. Each vinylferrocene sample was then added to an acrylamide-initiator mixture. Reaction mixtures were refluxed at 70° C. for 24 hours in nitrogen atmosphere. After cooling, the reaction mixtures were, separately, added drop-wisely to rapidly stirred acetone in order to precipitate a redox polymer. The precipitated redox polymer was washed with acetone and purified by multiple water-dissolving acetone-precipitating cycles. The purified product was then dried under vacuum at 50° C.

iii) Preparation of Poly(Vinyl Ferrocene-Co-Acrylamido-Sulphonic Acid) Polymers

Three samples containing 1.0 g acrylic acid dissolved in 10 ml of mixture solvent of ethanol/water (3 parts to 1 part) were prepared. A 0.30 ml aliquot of 0.10 g/ml oxygen-free persulfate solution was added to each sample after being deoxygenated for 10 minutes. 3 amounts of vinylferrocene ranging from 0.05 g to 0.16 g were dissolved in degassed ethanol to form 3 vinylferrocene solution samples, the amount of vinylferrocene that is added for each sample being calculated to obtain acrylamide-to-vinylferrocene feeding ratios (w/w) of 95:5, 90:10 and 85:15, respectively. Each vinylferrocene sample was then added to an acrylamide-initiator mixture. Reaction mixtures were refluxed at 70° C. for 24 hours in nitrogen atmosphere. After cooling, the reaction mixtures were, separately, added drop-wisely to rapidly stirred acetone in order to precipitate a redox polymer. The precipitated redox polymer was washed with acetone and purified by multiple water-dissolving acetone-precipitating cycles. The purified product was then dried under vacuum at 50° C.

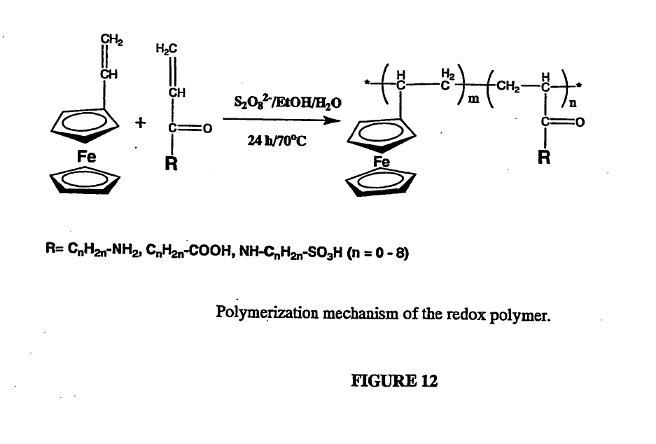

Co-polymerization of vinylferrocene with acrylamide and its derivatives were carried out based on conventional radical polymerization reaction. The general reaction equation is depicted in FIG. 12.

However, in order to successfully co-polymerize the monomers, great attention was given to the terminating effect of vinylferrocene in the system. Vinylferrocene usually acts as radical scavenger in the co-polymerization system. It was found that the amount of radical initiator is substantially less that these needed in normal polymerization systems. Higher amounts of radical initiator significantly reduced polymerization efficiency and the molecular weight of the product. Besides, the addition sequence also affects the polymerization efficiency.

Less than 20% of polymerization was observed when adding the persulfate radical initiator to the solution of vinylferrocene and acrylamide. This is probably because the formation of ferrocenium in the reaction mixture which resulted in the retardation of polymerization rate and much early termination of the polymer chain growth process. As shown in Table 1, under optimal conditions, relative high yields were obtained.

TABLE 1
Co-polymerization of vinylferrocene
and acrylamide and its derivatives

| Feeding ratio (w/w) | Yield (%) | VFc content (%) | Molecular weight |
|---|---|---|---|
| AA/VFc 95:5 | 80 | 4% | 3600 |
| AA/VFc 90:10 | 72 | 9% | 3100 |
| AA/VFc 85:15 | 56 | 11% | 2400 |
| AC/VFc 95:5 | 75 | 3% | 2800 |
| AC/VFc 90:10 | 55 | 7% | 2500 |
| AC/VFc 85:15 | 45 | 6% | 2000 |
| AAS/VFc 95:5 | 85 | 6% | 4000 |
| AAS/VFc 90:10 | 75 | 9% | 3500 |
| AAS/VFc 85:15 | 62 | 14% | 3000 |

However, the polymer yields decreased with increasing vinylferrocene feeding ratio, which indicated that the terminating effect in radical polymerization still exists, even though great care has already been taken in the polymerization process. It was also found that minute yields were obtained once the reaction mixture became blue, which was due to the formation of considerable amount of ferrocenium in the polymerization solution. Ferrocene loading varied from 3 to 14%, which is always less than ferrocene content in the monomer feedings.

Ferrocene loading in the redox polymer was determined from elemental analysis. Energy Dispersive X-ray Analysis (EDX) was used for this purpose. The energy of electron beam used on samples of the redox produced is 120 keV. The X-rays generated by the sample was subject to analysis by a lithium drifted silicon detector.

The molecular weight of the redox polymer was determined by gel permeation chromatography. Generally, the redox polymers prepared with higher ferrocene feeding ratio had lower molecular weight and broader molecular weight distribution.

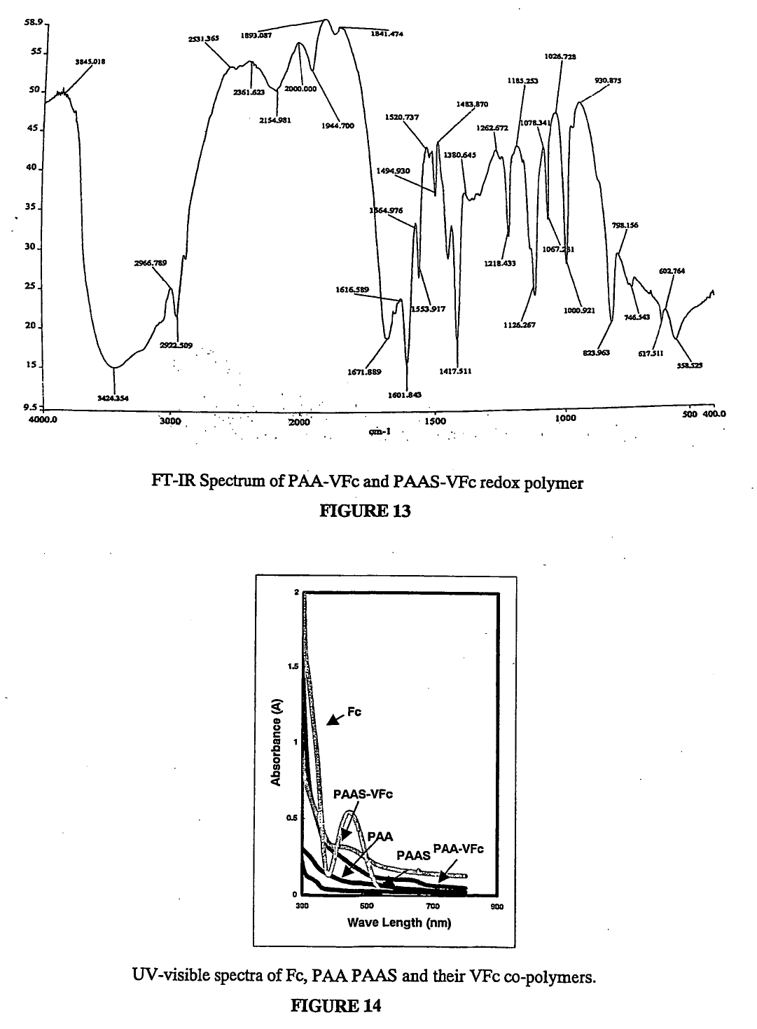

The synthesized copolymers were light-yellow colored, powdery materials. Molecular weights of the copolymers are in between 2000 and 4000 Daltons. FT-IR experiments (see FIG. 13) clearly showed the complete disappearance of vinyl absorption at 1650 suggesting that both acrylamide and vinylferrocene were successfully polymerized and the resulting redox polymer is of high purity, free of monomers. Further evidence can be found in the 1000-1300 cm⁻¹ region. Extremely strong adsorption accompanying by a weak one at 1126 cm⁻¹ indicates the presence of ferrocenyl units in the redox polymer and the strong absorption at 1218 cm⁻¹ suggested amide groups in the polymer. UV experiments, again, confirmed the successful co-polymerization of vinylferrocene and acrylamide. The minute shoulder at 300 is a clear indicative of ferrocene moiety in the co-polymer (see FIG. 13). Having ferrocenyl and amine or carboxylic acid moieties in the redox polymer rendered them with dual-function: redox activity for electron-mediating and chemical activity for cross-linking with proteins.

Increasing the feeding ratio of vinylferrocene was intended to increase the proportion of ferrocenyl moiety within the redox polymer. However, varying the amounts of vinylferrocene also affected the polymer yield. The highest yield obtained was when the vinylferrocene feeding ratio was the lowest, which is in good agreement with the unusual behavior of ferrocenyl compounds in radical polymerization. As indicated in Table 1, although the content of ferrocenyl moiety in the polymer increased with increasing vinylferrocene feeding ratio, but it is by far not linear at all. It was found that, for biosensing purpose, a vinylferrocene feeding ratio of 10% is sufficient, which gives good mediating function and good economy. The amount of initiator used in the polymerization also affected composition and yield of the redox polymer. It was found that good redox polymers were obtained when the initiator is in the range of 20-40 mg per gram of monomers.

### Example 5.2

### Obtaining Cyclic Voltamograms of the Redox Polymers

Redox polymers in phosphate-buffered saline (PBS) solutions in the presence of 0.0 μg, and 10 μg GOx, and 10 μg GOx and 10 mM glucose.

Electrochemical tests were performed with an AutoLab potentiostat/galvanostat running under the general purpose electrochemical system (GPES) manager version 4.9. A 3-electrode system cell, housed in a Faraday cage. The electrodes were a (Ag/AgCl) reference electrode, a platinum wire counter electrode and an Au working electrode (surface area of 7.94 mm²).

In contrast to vinylferrocene, the redox polymers that were synthesized have high solubility in water but are insoluble in most organic solvents. This characteristic renders the redox polymers ideal for uses as mediators in biosensing, particularly in enzyme-linked biosensing since most enzymes only work in aqueous media.

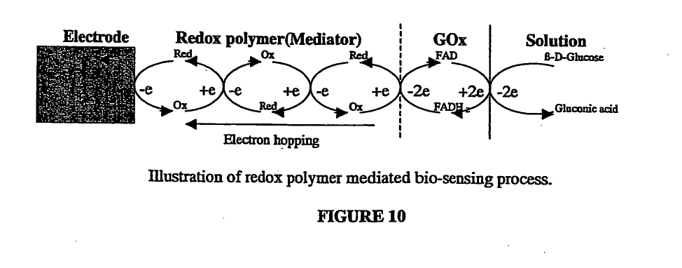

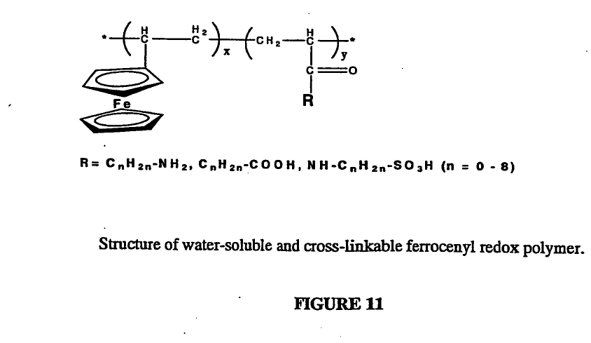

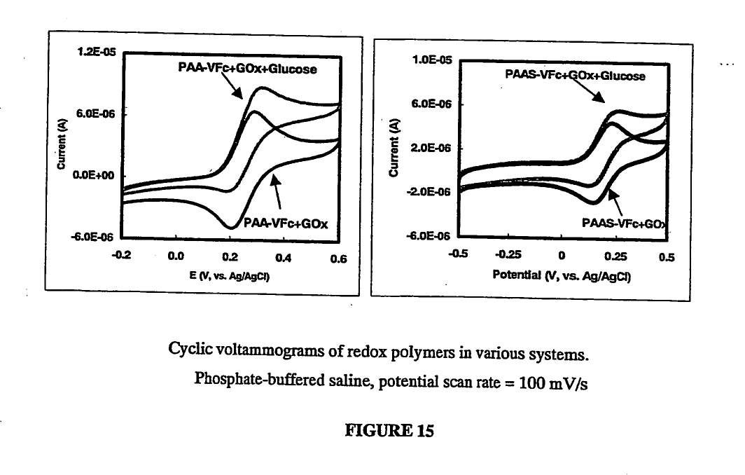

FIG. 15 shows typical cyclic voltamograms of the In PBS containing only the redox polymers, the voltamograms exhibited highly reversible solution electrochemistry: the redox waves centered at ˜0.18 V (vs. Ag/AgCl), the voltamogram has diffusion-limited shape, the magnitude of the anodic and cathodic peak current is the same, the peak-to-peak potential separation is 60 mV, very close to the theoretical value of 59 mV at 25° C. These redox waves can be assigned to the oxidation and reduction of ferrocenyl moieties in the redox polymers, which indicate excellent redox activity of the polymer. The voltametric experiments, again, demonstrated that vinylferrocene was successfully co-polymerized with acrylamide and its derivatives and the ferrocenyl moieties in the polymers retain their electroactivities. The redox polymers in PBS are in real solution form with free diffusional behavior. Spiking this solution with varies amounts of glucose did not change the voltamogram at all, which suggests that there is no catalytic oxidation of glucose by the redox polymers alone. Furthermore, no obvious changes were observed when adding small amounts of GOx in the redox polymer solution. The electrochemistry of the resulting solution was practically the same as the redox polymer alone solution. However, when 10 mM glucose was added to this solution, the enzymatic oxidation of glucose by GOx proceeds in the solution. The redox centers in GOx, FAD were converted to FADH₂. When the electrode potential was scanned past the redox potential of the redox polymer, significant amount of ferrocene moieties in the redox polymer was oxidized to ferrocenium near the electrode surface. The redox potential of FAD/FADH₂ in GOx is −0.36 V (vs. Ag/AgCl), which is much lower than the ferrocene/ferrocenium couple, the ferrocenium moieties in the vicinity of FADH₂ oxidize it back to FAD, and the ferrocenium moieties in the redox polymer are reduced to the original ferrocene moieties. These two reactions form a catalytic cycle, as illustrated in FIG. 10, or in other words, glucose oxidation by GOx is mediated by the redox polymer.

Thus, the catalytic reaction by the redox polymer greatly enhances the oxidation current in the solution containing glucose, as seen in FIG. 13 (light gray traces). If the electron-exchange among FADH₂, redox polymer and electrode are all very fast, large amount of ferrocenium moieties are produced during electrochemical oxidation, and they are, in turn, rapidly consumed by FADH₂. This is the reason for the much lower reduction current of ferrocenium moieties, as compared to that obtained in the glucose-free solution. These data suggests that the redox polymers function effectively as redox mediators in enzymatic reactions, shuttling electrons from the redox centers of enzyme to electrode surface.

### Example 5.3

### Synthesis of a Membrane Comprising Vinyl Ferrocene-Co-Acrylamide Cross-Linked with Glucose Oxidase-Bovine Serum Albumin (GOx-BSA)

The cross-linking reaction of the redox polymer with proteins was carried out to study the electrochemical properties of the resulting membrane. The enzyme GOx was used in the present example. Glutaradehyde and poly(ethylene glycol) diglycidyl ether (PEG) were chosen as cross-linkers. Biological grade glutaraldehyde (50% in water, product code 00867-1 EA) and poly(ethylene glycol) diglycidyl ether (PEGDE) (product code 03800) was obtained from Sigma-Aldrich.

First, poly(vinylferrocene-co-acrylamide) obtained from Example 5.1 was deposited onto a gold electrode. GOx-BSA was modified with the crosslinkers to provide GOx-BSA with an aliphatic carbon chain with a terminal aldehyde functional group, which can provide cross linkage with suitable functional groups on the immobilized mediator. Subsequently, the modified GOx-BSA was deposited and reacted with the immobilized initiator. The aldehyde group on the modified GOx-BSA reacted with the amine group on the PAA-VFc to form a covalent cross-linkages. After reaction was carried out, the PAA-VFc-GOx-BSA film was allowed to dry.

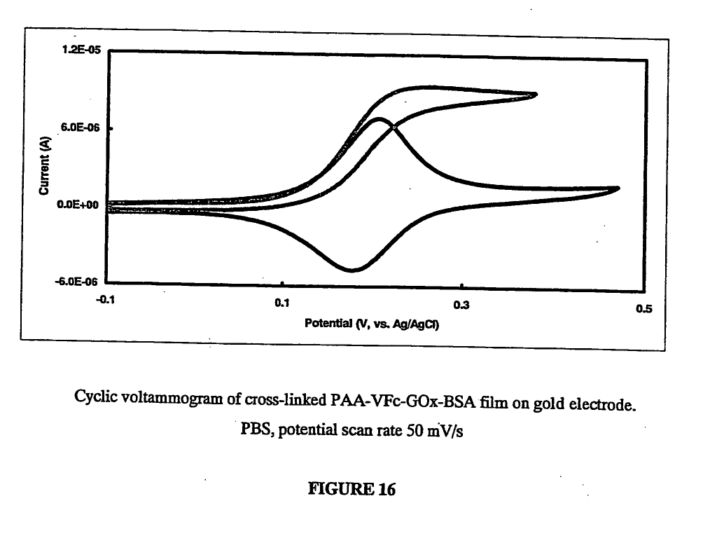

The cross-linked PAA-VFc-GOx-BSA film on gold electrode was subjected to voltametry analysis. Blank PBS was used, and a potential scan rate of 50 mV/s was applied. FIG. 16 shows a cyclic voltamogram of the PEG cross-linked PAA-VFc with GOx and BSA on gold electrode in blank PBS. As illustrated in FIG. 16, the cross-linked film exhibited exactly as expected for a highly reversible surface immobilized redox couple (A. J. Bard, L. R. Faulkner, Electrochemical Methods, John Wiley & Sons: New York, 2001.) with little change after exhaustive washing with water and PBS, and after numerous repetitive potential cycling between −0.2 V and +0.8V, revealing a highly stable surface immobilized ferrocenyl film on gold electrode. At slow scan rates, <100 mV/s, a remarkably symmetrical signal was recorded as expected for a surface confined one-electron redox system exhibiting an ideal Nemstian behavior: The peak current is proportional to the potential scan rate, the peak-to-peak potential separation is much less than 59 mV, as observed in the case of diffusional behavior in solution (see FIG. 15), and the width of the current at half-peak height is around 90 mV. Such results ascertain that all of the ferrocenyl redox centers are allowed to reach the electrode surface and proceed to reversible heterogeneous electron transfer. Upon adding 10 mM of glucose to the PBS solution, a typical catalytic electrochemical curve was obtained However, the reduction peak of the redox polymer disappeared (FIG. 16, gray trace). This meant that the sensing layer was homogenously maintained in the reduced state by the transfer of electrons from the reduced GOx to the ferrocenyl moieties. The rapid response and current detected indicated excellent mediating function of the redox polymer the high current sensitivity (750 nA/mM glucose) of the biosensors.

1. A method for the electrochemical detection of an analyte molecule by means of a detection electrode, the method comprising:
(a) immobilizing capture molecules, which are capable of binding the analyte molecule to be detected, on the detection electrode;
(b) contacting the electrode with a solution supposed to contain the analyte molecule to be detected;
(c) allowing the analyte molecule contained in said solution to bind to the capture molecules on the electrode, thereby allowing formation of complexes between a capture molecule and an analyte molecule, said complexes forming a first layer on the detection electrode;
(d) contacting the detection electrode with an electrochemical activator, wherein said electrochemical activator has a electrostatic net charge that is complementary to the electrostatic net charge of the complex formed between a capture molecule and an analyte molecule, thereby forming a second layer on the electrode, wherein the second layer and the first layer together form a conducting bilayer;
(e) contacting the detection electrode with an agent capable of transferring electrons to or from the electrochemical activator from or to the electrode, respectively; performing an electrical measurement at the detection electrode, and;
(g) detecting the analytes by comparing the result of the electrical measurement obtained with that of a control measurement.

2. The method of claim 1, wherein the electrochemical activator is a polymeric redox mediator capable of transferring electrons between the analyte and the electrode.

3. The method of claim 2, wherein the electrochemical activator comprises metal ions.

4. The method of claim 3, wherein the metal ions are selected from the group consisting of silver, gold, copper, nickel, iron, cobalt, osmium, ruthenium, and mixtures thereof.

5. The method of claim 4, wherein the electrochemical activator is selected n-phosphonic acid, wherein n=0-12.

6. The method of claim 1, wherein the agent capable of transferring electrons to or from the electrochemical activator is an enzyme or an enzyme-conjugate.

7. The method of claim 6, wherein the enzyme is an oxidoreductase or a mixture of oxidoreductases.

8. The method of claim 7, wherein the oxidoreductase is selected from the group consisting of glucose oxidase, hydrogen peroxidase, lactate oxidase, alcohol dehydrogenase, hydroxybutyrate dehydrogenase, lactic dehydrogenase, glycerol dehydrogenase, sorbitol dehydrogenase, glucose dehydrogenase, malate dehydrogenase, galactose dehydrogenase, malate oxidase, galactose oxidase, xanthine dehydrogenase, alcohol oxidase, choline oxidase, xanthine oxidase. choline dehydrohenase, pyruvate dehydrogenase, pyruvate oxidase, oxalate oxidase, bilirubin oxidase, glutamate dehydrogenase, glutamate oxidase, amine oxidase, NADPH oxidase, urate oxidase, cytochrome C oxidase, and actechol oxidase.

9. The method of claim 1, wherein the capture molecules are capable of specifically binding the analytes to be detected.

10. The method of claim 1, wherein the analyte to be detected is selected from the group consisting of nucleic acids, oligonucleotides, proteins, peptides, oligosaccharides, polysaccharides and complexes thereof.

11. The method of claim 10, wherein the analyte to be detected is a nucleic acid molecule.

12. The method of claim 11, wherein the nucleic acid molecule has a pre-defined sequence.

13. The method of claim 12, wherein the nucleic acid molecule comprise at least one single-stranded region.

14. The method of claim 13, wherein the capture molecule is at least one nucleic acid probe having a sequence complementary to a single-stranded region of the nucleic acid molecule to be detected.

15. The method of claim 10, wherein the analyte to be detected is a protein or a peptide.

15. The method of claim 15, wherein the capture molecule is at least on ligand capable of binding proteins or peptides.

16. The method of claim 1, wherein a blocking agent is immobilized on the electrode prior to contacting the electrode with the solution supposed to contain the analyte molecule.

17. A method for the electrochemical detection of an analyte molecule by means of a detection electrode, the method comprising:
(a) immobilizing capture molecules, which are capable of binding the analyte molecule to be detected, on the detection electrode;
(b) contacting the electrode with a solution supposed to contain the analyte molecule to be detected;
(c) allowing the analyte molecule contained in said solution to bind to the capture molecules on the electrode, thereby allowing formation of complexes between a capture molecule and an said complexes forming a first layer on the detection electrode;
(d) contacting the detection electrode with an electrochemical activator, wherein said electrochemical activator has an electrostatic net charge that is complementary to the electrostatic net charge of the complex formed between a capture molecule and an analyte molecule, thereby forming a second layer on the electrode, wherein the second layer and the first layer together form a conducting bilayer, and wherein the capture molecules are capable of transferring electrons to or from the electrochemical activator from or to the electrode, respectively;
(e) performing an electrical measurement at the detection electrode, and; detecting the analytes by comparing the result of the electrical measurement obtained with that of a control measurement.

18. An electrode arrangement, comprising a detection electrode, suitable for carrying out an electrochemical detection of an analyte molecule as defined in claim 1, comprising:
(a) a first layer on the detection electrode comprising complexes between a capture molecule, which is capable of binding the analyte molecule to be detected, and an analyte molecule; and
(b) a second layer comprising an electrochemical activator, wherein said electrochemical activator has an electrostatic net charge that is complementary to the electrostatic net charge of the complex formed between a capture molecule and an analyte molecule, wherein the second layer and the first layer together form a conducting bilayer.

19. The electrode arrangement of claim 18, wherein the electrochemical activator is a polymeric redox mediator capable of transferring electrons between the analyte and the electrode.

20. The electrode arrangement of claim 19, wherein the agent for increasing conductivity of the analytes contains metal ions.

21. The electrode arrangement of claim 20, wherein the metal ions are selected from the group consisting of silver, gold, copper, nickel, iron, cobalt, osmium, ruthenium and mixtures thereof.

22. The electrode arrangement of claim 18, further comprising an agent capable of transferring electrons to or from the polymeric redox mediator from or to the electrode, respectively, wherein the agent is bound to, intercalated in or associated with the conducting bilayer

23. The electrode arrangement of claim 22, wherein the agent is an enzyme or an enzyme-conjugate.

24. Use of an electrode arrangement of claim 18 as biosensor.

25. A biosensor for the electrochemical detection of an analyte molecule, comprising:
(a) an detection electrode;
(b) a first layer on the detection electrode comprising complexes between a capture molecule, which is capable of binding the analyte molecule to be detected, and an analyte molecule; and
(c) a second layer comprising an electrochemical activator, wherein said electrochemical activator has an electrostatic net charge that is complementary to the electrostatic net charge of the complex formed between a capture molecule and an analyte molecule, wherein the second layer and the first layer together form a conducting bilayer.

26.-45. (canceled)


SEQUENCE LISTING (14 sequences)

SEQ ID NO: 1 - DNA, 18 bp, Artificial
  Primer
atggtgaaggtcggtgtc

SEQ ID NO: 2 - DNA, 20 bp, Artificial
  Primer
ttactccttggaggccatgt

SEQ ID NO: 3 - DNA, 20 bp, Artificial
  Primer
atggaggattcacagtcgga

SEQ ID NO: 4 - DNA, 18 bp, Artificial
  Primer
tcagtctgagtcaggccc

SEQ ID NO: 5 - DNA, 35 bp, Artificial
  Capture probe
ttttttttttttttactccttggaggccatgtagg

SEQ ID NO: 6 - DNA, 35 bp, Artificial
  Capture probe
ttttttttttttatggtgaaggtcggtgtcaacgg

SEQ ID NO: 7 - DNA, 32 bp, Artificial
  Capture probe
  misc_feature (1)..(): XXX
ttttttttttttatggaggattcacagtcgga

SEQ ID NO: 8 - DNA, 32 bp, Artificial
  Capture probe
tttttttttttttcagtctgagtcaggcccca

SEQ ID NO: 9 - DNA, 35 bp, Artificial
  Capture probe
ttttttttttttcctctcgcgagtcaacagaaacg

SEQ ID NO: 10 - DNA, 50 bp, Artificial
  Capture probe
gccagcgttcaatctgagccatgatcaaactcttcaaaaaaaaaaaaaaa

SEQ ID NO: 11 - DNA, 32 bp, Artificial
  Detection probe
aaaaaaaaaaaaaagctgcctcccgtaggagt

SEQ ID NO: 12 - DNA, 71 bp, Artificial
  Oligonucleotide
aaattgaagagtttgatcatggctcagattgaacgctggcaaaaaaaaaaaaaactcctacgggaggcagc

SEQ ID NO: 13 - DNA, 71 bp, Artificial
  Oligonucleotide
aaattgaagagtttgatcatgtctcagattgaacgctggcaaaaaaaaaaaaaactcctacgggaggcagc

SEQ ID NO: 14 - DNA, 71 bp, Artificial
  Oligonucleotide
aaattgaagagtatgatcatgtctcagattgaacgctggcaaaaaaaaaaaaaactcctacgggaggcagc